(19) United States
(12) Patent Application Publication
(10) Pub. No.: US 2025/0113976 A1
(43) Pub. Date: Apr. 10, 2025

(54) Footwear Treatment System

(71) Applicant: Sensor Electronic Technology, Inc., Columbia, SC (US)

(72) Inventor: Piyush Sharma, Columbia, SC (US)

(21) Appl. No.: 18/896,251

(22) Filed: Sep. 25, 2024

Related U.S. Application Data
(60) Provisional application No. 63/588,463, filed on Oct. 6, 2023.

(51) Int. Cl. A47L 23/02; A47L 23/18; A47L 23/20; A61L 2/08; A61L 2/10; A61L 2/24

(52) U.S. Cl. CPC A47L 23/02; A47L 23/20; A61L 2/088; A61L 2/10; A61L 2/24; A47L 23/18; A61L 2202/11; A61L 2202/14

(57) ABSTRACT

A footwear treatment system can include a pair of elongate supports, each of which can include a cylindrical footwear extension region configured to be inserted into and hold a footwear item. The system can emit ultraviolet light that is directed into an interior of the footwear item using the corresponding elongate support. The system also can circulate air within the interior of the footwear item using the corresponding elongate support and filter the circulating air. As a result of the treatment, one or more microorganisms and/or compounds associated with odor can be reduced to an acceptable level.

### REFERENCE TO RELATED APPLICATIONS

The current application claims the benefit of U.S. Provisional Application No. 63/588,463, filed on 6 Oct. 2023, which is hereby incorporated by reference.

### TECHNICAL FIELD

The disclosure relates generally to treating footwear, and more particularly, to a solution for deodorizing footwear.

### BACKGROUND ART

Currently, shoe deodorization mainly involves using sprays, powders, and insoles. However, these deodorization methods only provide temporary solutions and do not address the root cause of shoe odor, which is the proliferation of bacteria and moisture inside the shoe.

Some existing alternative proposals to deodorize shoes include using UV-C light, activated charcoal, and other types of air purifiers. However, these existing alternatives have drawbacks. For example, UV-C light can be harmful to human skin and eyes, and may not effectively reach all areas inside the shoe. Activated charcoal also has limited effectiveness and needs to be replaced regularly. Other air purifiers may be too bulky or expensive for everyday use in a home setting.

### SUMMARY OF THE INVENTION

Aspects of the invention provide a solution for deodorizing footwear as described herein. Embodiments can provide a footwear treatment system that includes a pair of elongate supports, each of which can include a cylindrical footwear extension region configured to be inserted into and hold a footwear item. The system can emit ultraviolet light that is directed into an interior of the footwear item using the corresponding elongate support. The system also can circulate air within the interior of the footwear item using the corresponding elongate support and filter the circulating air. As a result of the treatment, one or more microorganisms and/or compounds associated with odor can be reduced to an acceptable level.

Embodiments provide shoe deodorizing using ultraviolet light. The ultraviolet light can comprise, for example, ultraviolet-C (UVC) light.

Embodiments can use a light source in conjunction with a photocatalyst. For example, embodiments can use a combination of ultraviolet-A (UVA) light and a photocatalyst, such as titanium dioxide (Ti0₂).

Embodiments can include a replaceable odor absorber cartridge, such as activated charcoal, baking soda, and/or the like.

Embodiments can include an air movement component, such as one or more fans, to circulate air within the shoe during deodorization. A fan can be located in each shoe or located at another location to facilitate airflow within the shoe.

Embodiments can include a photo catalyst cartridge and/or an odor absorber cartridge, which can be placed within a path of the circulating air, and can be configured for easy removal, e.g., for rinsing, replacement, and/or the like.

Embodiment can include one or more hollow shoe inserts configured to suspend shoes for storage while simultaneously allowing for airflow to remove moisture and treat odors. Embodiments of a hollow shoe insert can comprise a cylindrical rod. Other embodiments can include supporting rods or tubing to channel air to the shoes.

To ensure safe usage, embodiments can include a proximity sensor, which can be used to turn on and/or off the system. For example, the proximity sensor can detect when a shoe is placed and/or removed from a corresponding insert.

Embodiments can comprise a shoe cabinet, within which the shoe inserts can be located. The shoe cabinet can be configured to prevent the emission of ultraviolet radiation therefrom. A sensor can detect when the shoe cabinet is closed and/or opened and turn the system on and/or off in response thereto.

Overall, a solution described herein provides a comprehensive safe, effective, and convenient solution for shoe deodorization, which can be customized to meet specific user needs.

A first aspect of the invention provides a footwear treatment system comprising: a pair of elongate supports, each elongate support including a cylindrical footwear extension region configured to be inserted into and hold a footwear item in an approximately vertical orientation; at least one ultraviolet light source configured to emit ultraviolet light directed into an interior of the footwear item using the corresponding elongate support; an air circulation component configured to circulate air within the interior of the footwear item using the corresponding elongate support; and at least one filtration component configured to filter the circulating air.

A second aspect of the invention provides a footwear treatment system comprising: an elongate support structure including a plurality of elongate supports, wherein each elongate support is configured to support one of a plurality of footwear items; at least one ultraviolet light source configured to emit ultraviolet light directed into an interior of each of the plurality of footwear items using the elongate support structure; an air circulation component for circulating air within the interior of each of the plurality of footwear items using the elongate support structure; and a computing unit configured to control operation of the at least one light source and the air circulation component.

A third aspect of the invention provides a method of treating footwear, the method comprising: circulating with a fan air through a pair of hollow cylindrical elongate support, each of which is supporting a footwear item in an approximately vertical orientation; emitting ultraviolet light from at least one ultraviolet light source into an interior of each footwear item using the corresponding elongate support; and controlling the circulating and emitting based on at least one of: an amount of time, a treatment dose, a detected presence of a microorganism or a compound associated with odor, a detected humidity level, or a detected status of a door to a treatment housing in which the elongate support is located.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows an illustrative footwear treatment system according to an embodiment.

FIG. 2 shows another illustrative footwear treatment system according to an embodiment.

FIG. 3 shows an illustrative embodiment of a portion of a footwear support structure according to another embodiment.

FIG. 4 shows another illustrative footwear treatment system according to an embodiment.

FIG. 5 shows an illustrative flow diagram according to embodiments.

FIG. 6 shows still another illustrative footwear treatment system according to an embodiment.

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As discussed above, aspects of the invention provide a solution for deodorizing footwear as described herein. Embodiments can provide a footwear treatment system that includes a pair of elongate supports, each of which can include a cylindrical footwear extension region configured to be inserted into and hold a footwear item. The system can emit ultraviolet light that is directed into an interior of the footwear item using the corresponding elongate support. The system also can circulate air within the interior of the footwear item using the corresponding elongate support and filter the circulating air. As a result of the treatment, one or more microorganisms and/or compounds associated with odor can be reduced to an acceptable level.

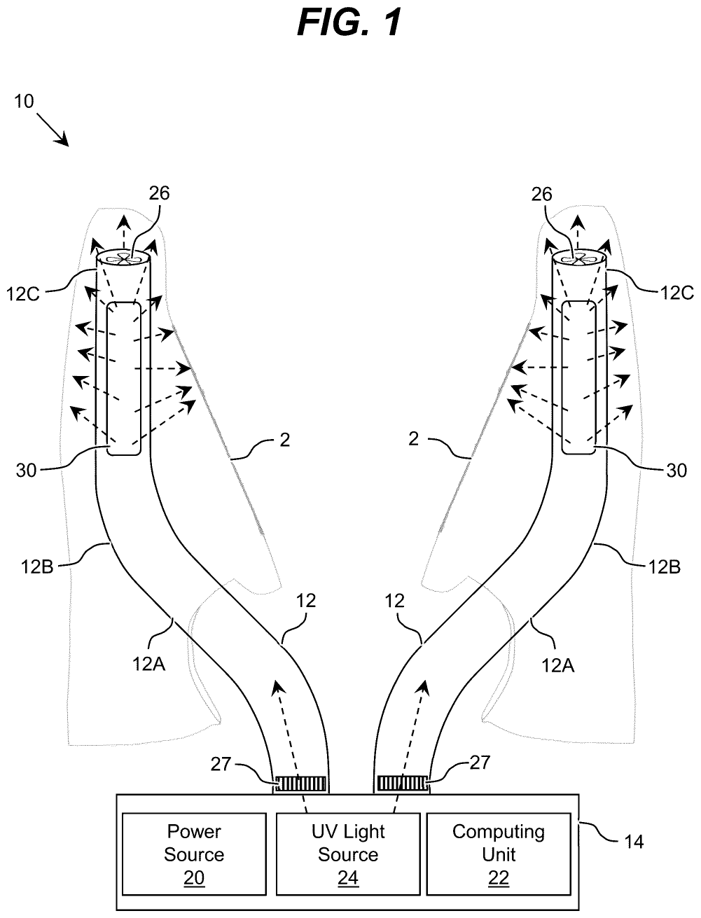

FIG. 1 shows an illustrative footwear treatment system 10 according to an embodiment. Illustrative types of footwear include any of various types of sneakers, shoes, socks, boots, etc. In embodiments, the footwear treatment system 10 is configured to deodorize any of various types of footwear 2. Furthermore, the footwear treatment system 10 can be configured to remove moisture from the footwear 2.

Regardless, embodiments include at least one pair of footwear support structures 12, each of which is configured to support the footwear 2 during treatment. As illustrated, a footwear support structure 12 can comprise an elongate member that includes an insertion region 12A, a curved region 12B, and a footwear extension region 12C. As illustrated, the footwear extension region 12C can be oriented such that a corresponding footwear 2 is held in an approximately vertical orientation. Each region 12A-12C can be configured to facilitate insertion of the footwear extension region 12C into a desired footwear 2 item, without requiring significant stretching, bending, pulling, etc., of the footwear 2. To this extent, embodiments of the treatment system 10 can include various alternative sizes and configurations of footwear support structures 12 to accommodate footwear of various types and sizes.

Each footwear support structure 12 can be supported by a base housing 14, which can include one or more additional components of the footwear treatment system 10 described herein. To this extent, the base housing 14 is shown including a power source 20, a computing unit 22, and an ultraviolet light source 24. The power source 20 can comprise any type of power source. For example, the power source 20 can comprise one or more batteries, an electrical plug and transformer for obtaining power from an electrical receptacle, and/or the like. Regardless, the power source 20 can be configured to provide sufficient power to operate the remaining components of the footwear treatment system 20.

The computing unit 22 can comprise any type of computing unit capable of controlling operation of one or more input and/or output devices in order to perform a treatment on the footwear 2. Embodiments of the computing unit 22 can include a programmable computer (e.g., including one or more processors, memory, and I/O interfaces) executing program code installed thereon, one or more special purpose microprocessors, and/or the like.

The computing unit 22 can be configured to control operation of an ultraviolet light source 24. The ultraviolet light source 24 can be configured to generate ultraviolet light, which is directed to perform a desired treatment on the footwear 2. Embodiments of the ultraviolet light source 24 can include one or more ultraviolet light emitting diodes, one or more mercury-based lamps, one or more fluorescent lamps, and/or the like. In embodiments, the ultraviolet light emitted by the ultraviolet light source 24 is directed into an interior of the footwear 2, e.g., using the footwear support structure 12.

The ultraviolet light emitted by the ultraviolet light source 24 can comprise any type of ultraviolet light that is suitable for providing a desired treatment. In embodiments, the ultraviolet light comprises ultraviolet-C light, which has a peak wavelength in a range between 100 nanometers and 280 nanometers. In this case, the ultraviolet light can be configured to kill or inactivate microorganisms that may be present within the footwear 2. In embodiments, the ultraviolet light comprises ultraviolet-A light, which has a peak wavelength in a range between 315 nanometers and 400 nanometers.

In some embodiments, the footwear support structure 12 can include an interior that is configured to provide for the transmission of ultraviolet light from the ultraviolet light source 24 to one or more emission locations on the footwear extension region 12C and/or the curved region 12B of the footwear support structure 12. For example, the footwear support structure 12 can comprise a hollow interior, at least a portion of which can include a reflective surface that guides the ultraviolet light emitted from the ultraviolet light source 24 to one or more desired emission locations within the footwear 2 interior. In embodiments, the footwear support structure 12 can include one or more physical light guiding structures, such as optical fibers and/or the like, which can be configured to guide the ultraviolet light emitted from the ultraviolet light source 24 to one or more desired emission locations within the interior of the footwear 2.

In embodiments, one or more portions of the footwear support structure 12 includes an exterior surface which is configured to allow ultraviolet light to be emitted therefrom. For example, as illustrated, ultraviolet radiation can be emitted from an end of the footwear support structure 12. Additionally, the footwear extension region 12C can include one or more emission windows 30 through which ultraviolet light can be emitted into an interior of the footwear 2. The emission window 30 can comprise an opening in the footwear extension region 12C through which ultraviolet light guided up into the footwear extension region 12C can be emitted. Alternatively, the emission window 30 can be formed from a material that allows ultraviolet radiation to pass, e.g., from a hollow interior, there through. Illustrative materials include fused quartz, UVC silicones, clear acrylic, and/or the like. Embodiments of the emission window 30 can be configured to cause the ultraviolet radiation to be emitted in numerous directions in order to provide ultraviolet illumination throughout most of an interior region of the footwear 2. To this extent, embodiments of the emission window 30 can include surface roughening and/or the like, which can cause ultraviolet radiation to be scattered in different directions after passing through the surface.

Embodiments can include one or more additional components. For example, each footwear support structure 12 is also shown including a fan 26, which is illustrated as being located at an internal end of the footwear support structure 12. The fan 26 can be configured to circulate air within an interior of the footwear 2. Such air circulation can assist in drying out an interior of the footwear 2, increase circulation of microorganisms which can be treated with the ultraviolet radiation, etc.

In embodiments, the fan 26 can be configured to direct air through the footwear support structure 12 and into the footwear 2. In this case, the footwear support structure 12 can be configured to pull the air from one or more locations internal or external to the footwear 2, e.g., from a set of openings 27 located adjacent to the base housing 14, from an opening through which ultraviolet light is emitted, and/or the like. In other embodiments, the fan 26 can be configured to direct air from the footwear 2 into the hollow interior of the footwear support structure 12. In this case, the footwear support structure 12 can be configured to vent the air from one or more locations, which can be located internal and/or external to the footwear 2.

The computing unit 22 can be configured to operate the ultraviolet light source 24 and each fan 26 using any solution. For example, after footwear 2 is placed on the footwear support structure 12, the computing unit 22 can turn on the fans 26 to allow air to circulate within the footwear 2. The fans 26 can remain on for a predetermined period of time, as long as the footwear 2 remains on the footwear support structure 12. Similarly, the computing unit 22 can turn on the ultraviolet light source 24 in response to the footwear 2 being placed on the footwear support structure 12. The computing unit 22 can operate the ultraviolet light source 24 in any manner to deliver a desired dose of ultraviolet light to the interior of the footwear 2, as long as the footwear 2 remains on the footwear support structure. For example, the computing unit 22 can operate the ultraviolet light source 24 to generate ultraviolet light in a pulsed manner, continuously, for multiple predetermined periods of time separated by predetermined periods of time when the ultraviolet light sources are off, and/or the like.

As illustrated in FIG. 1, embodiments of the system 10 can include a single ultraviolet light source 24, which generates ultraviolet light for multiple footwear support structures 12. Regardless, it is understood that this is only illustrative and other configurations are possible. For example, embodiments of the footwear support structure 12 can include one or more light sources (e.g., light emitting diodes, exposed ends of light guiding structures, etc.) from which ultraviolet light can be emitted into an interior of the footwear 2. In this case, the footwear support structure 12 can include any required wiring, light guiding structure, and/or the like, embedded therein.

In embodiments, the system 10 can be configured and/or can include one or more components that prevent ultraviolet light from being directed into a footwear support structure 12 when footwear 2 is not present thereon. For example, the UV light source 24 can include a plurality of individually operable light sources, each of which is configured to emit light directed into one of the footwear support structures 12. The computing unit 22 can be configured to only activate the individually operable light sources that emit ultraviolet light into the footwear support structure(s) 12 on which footwear 2 is located.

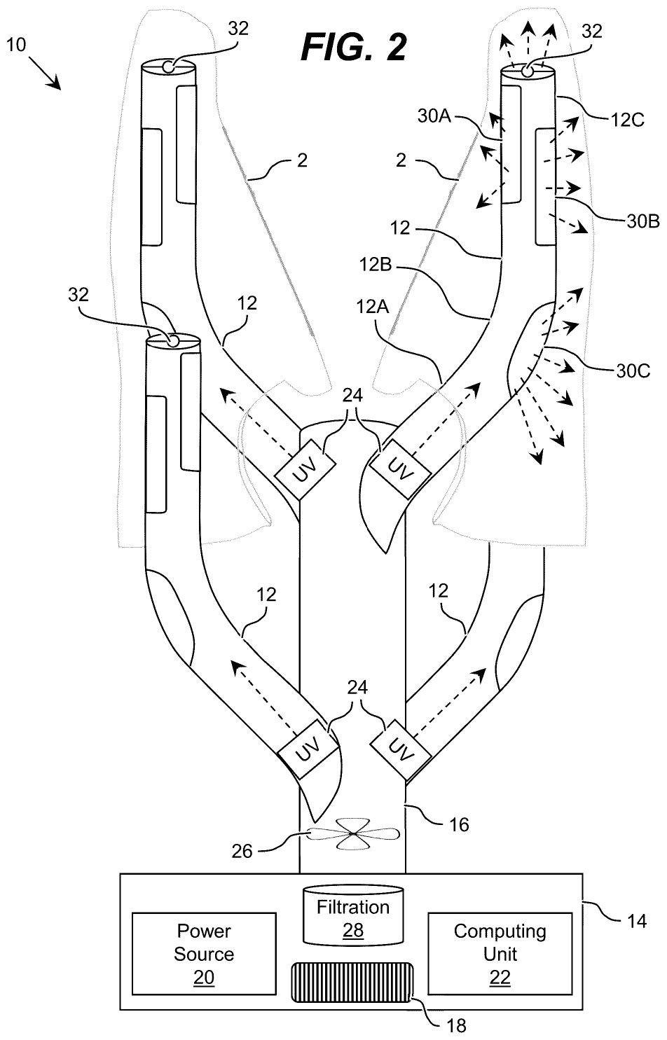

FIG. 2 shows another illustrative footwear treatment system 10 according to an embodiment. In this case, the footwear treatment system 10 can concurrently treat more than one pair of footwear 2. To this extent, the footwear treatment system 10 is shown including four footwear support structures 12, each of which is mounted to a central structure 16, which is supported by a base housing 14.

Each footwear support structure 12 is shown including its own ultraviolet light source 24. In embodiments, the computing unit 22 can operate the ultraviolet light source 24 for a footwear support structure 12 in response to determining that footwear 2 has been placed thereon. To this extent, each footwear support structure 12 is further shown including a footwear sensor 32, which can detect when footwear 2 is present or not present on the footwear support structure 12. The footwear sensor 32 can comprise any type of sensor, such as a proximity sensor, an environmental sensor, and/or the like. Regardless, in response to receiving a signal from the footwear sensor 32 that an object, such as footwear 2, is located thereon, the computing unit 22 can turn on the corresponding ultraviolet light source 24 to perform a treatment described herein.

While each footwear support structure 12 is shown including a single footwear sensor 32 for illustrative purposes, it is understood that multiple sensors can be implemented on a footwear support structure 12, a single footwear sensor 32 can detect footwear on multiple footwear support structures 12, and/or the like. For example, as illustrated, the footwear support structure 12 can include multiple windows 30A-30C from each of which ultraviolet light can be emitted when the ultraviolet light source 24 is on. In embodiments, each window 30A-30C (along with an end of the footwear support structure 12) can have a corresponding sensor 32 associated therewith. In this case, the computing unit 22 can only turn on the ultraviolet light source 24 in response to all of the sensors indicating that an object is located nearby. Alternatively, each window 30A-30C can comprise a corresponding shade mechanism, such as a damper, which can be opened and closed by the computing unit to prevent ultraviolet light from being emitted therefrom when no object is sensed as being located adjacent thereto. In this manner, ultraviolet light is less likely to be emitted into an ambient environment. In other embodiments, a footwear sensor 32 can comprise a camera or the like, which can acquire image data that the computing unit 22 can process to determine which of the corresponding footwear support structures 12 includes footwear 2 located thereon.

The system 10 of FIG. 2 also is shown including a single fan 26. In this case, each of the footwear support structures 12 and the central structure 16 can have hollow interiors. When footwear 2 is located on one or more of the footwear support structures 12, the computing unit 22 can turn on the fan 26 to draw air from one or more of the footwear support structures 12 into the central structure 16. In embodiments, the computing unit 22 can operate one or more valves, such as a damper, to selectively close (when no footwear is present) and open (when footwear is present) air flow through a footwear support structure 12 due to operation of the fan 26.

In embodiments, the system 10 can include a filtration component 28, which can filter the air drawn out of the footwear 2. The filtration component 28 can comprise any type of filter, such as an activated charcoal filter, a baking soda-based filter, a HEPA filter, a carbon filter, and/or the like, which can absorb water and/or molecules that cause odor. After being filtered, the air can exit into ambient through one or more vents 18. A filtration component 28 may periodically require cleaning, replacement, and/or the like. To this extent, the computing unit 22 can monitor an effectiveness of the filtration component 28, e.g., by monitoring the air flow there through, and indicate to a user when the filtration component 28 requires cleaning or replacement. The housing 14 can include any type of access, such as a door, an opening, and/or the like, which enables a user to remove and insert the filtration component 28 in the corresponding location.

While each footwear support structure 12 is shown having substantially the same configuration, it is understood that embodiments can include footwear support structures 12 having different configurations, generally in pairs, to accommodate different items of footwear. Such different configurations can include different sizes, different lengths for the insertion region 12A and/or the footwear extension region 12C, etc. Additionally, a footwear support structure 12 can be configured to treat an item of footwear 2 that does not have a closed end, such as a sandal, or the like. In this case, ultraviolet light can be emitted only from a side of the footwear support structure 12 located adjacent to a bottom of the sandal.

Embodiments of the central structure 16 can enable the selective attachment of different footwear support structures 12 of varying configurations through the use of a removable fastening mechanism, such as a threaded mount, a magnetic mount, a transition fit, and/or the like. When a footwear support structure 12 is removable, the computing unit 22 can receive data from one or more sensors located at the mounting location indicating the footwear support structure 12 is or is not present. The computing unit 22 can adjust operation of the system 10 based on one or more of the footwear support structures 12 not being present.

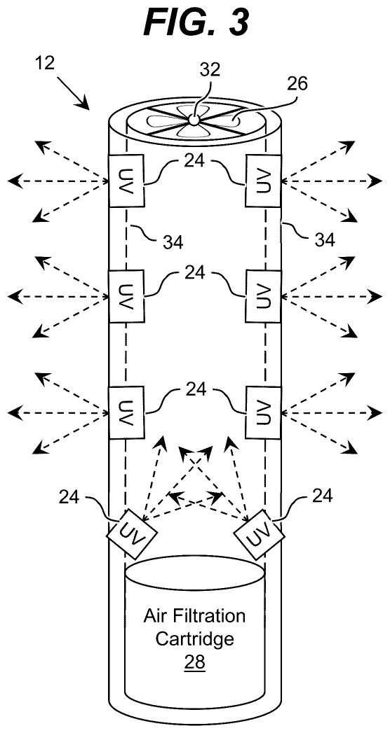

FIG. 3 shows an illustrative embodiment of a portion of a footwear support structure 12 according to another embodiment. In this case, the footwear support structure 12 is shown including multiple ultraviolet light sources 24 spaced along a length of the footwear support structure 12, each of which can emit ultraviolet light directed onto footwear located adjacent thereto. Furthermore, the footwear support structure 12 is shown having a hollow interior, with ultraviolet light sources 24 configured to emit ultraviolet light within the hollow interior. In embodiments, the ultraviolet light sources 24 can comprise one or more light emitting devices, such as LEDs, locations from which ultraviolet light can be emitted, such as ends of optical fiber, and/or the like.

In embodiments, an interior surface and/or an exterior surface of the footwear support structure 12 can be configured to assist with treating the footwear. For example, embodiments of the support structure 12 can include one or more surfaces coated with a photocatalyst 34, such as titanium dioxide. When illuminated with ultraviolet light, the photocatalyst can assist with purification of the surrounding environment. To this extent, embodiments can include a photocatalyst 32 coating on the footwear extension region 12C of a footwear support structure 12 described herein.

Embodiments of the footwear support structure 12 can include one or more of various other types of sensors, each of which can provide data for use by the computing unit 22 in treating the footwear. Illustrative sensors can include a humidity sensor, which can detect a level of moisture within the footwear, a visible light sensor, which can detect fluorescence within the footwear in response to illumination by ultraviolet radiation, a chemical or gas sensor, which can detect amounts of one or more types of chemicals responsible for odor, and/or the like.

Furthermore, the footwear support structure 12 is shown including a fan 26, a filtration cartridge 28, and a sensor 32, each of which can be operated as described herein. When included in a footwear support structure 12, it is understood that a mechanism can be included to enable access to and removal/replacement of the filtration cartridge 28 by a user.

The footwear support structure 12 of FIG. 3 is intended only to illustrate different features that can be implemented in embodiments. When the footwear support structure 12 is removable and/or replaceable, it is understood that a mounting location for the footwear support structure 12 can include an interface with one or more mechanisms for enabling communications and/or control between the various components located in the footwear support structure 12 and the computing unit 22.

For example, at the connection point, one or more pins, connectors, and/or the like, can enable communications between the computing unit 22 and the components located within the footwear support structure 12.

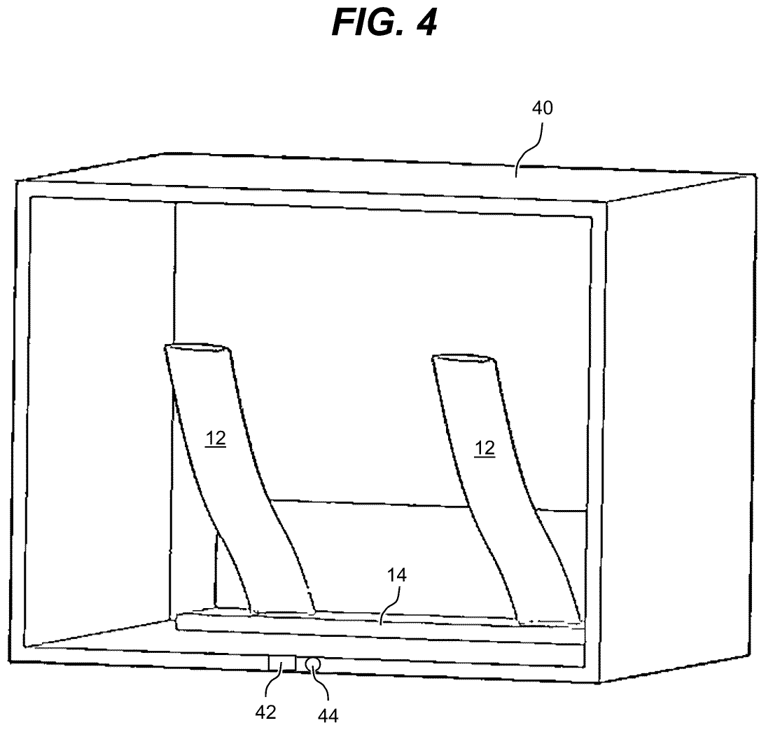

FIG. 4 shows another illustrative footwear treatment system 10 according to an embodiment. In this case, each footwear support structure 12 and some or all of the housing 14 can be located within a larger treatment housing 40. The treatment housing 40 can be configured to contain ultraviolet radiation and provide a contained environment in which treatment of footwear can be performed. To this extent, while not shown for clarity, the treatment housing 40 can include a front door, which can be secured shut by a securing mechanism 42, such as a latch, a catch, or the like. The treatment housing 40 also can include one or more sensors 44, such as a proximity sensor, which can detect when the door is open or closed, and provide such information for use by the computing unit 22 in treating the footwear. An exterior of the treatment housing 40 can include one or more input and/or output devices, which can enable a user to selectively request treatment, view a progress of treatment, view any warnings or errors, such as a required filter replacement, a low battery, and/or the like, etc.

When the treatment housing 40 is included, an interior of the treatment housing 40 can be fabricated with any type of material, which can prevent ultraviolet radiation from escaping therefrom. Such material can be ultraviolet absorbing to prevent over radiating an exterior surface of the footwear. Additionally, some or all of the interior of the treatment housing 40 can be configured to treat the air located therein, e.g., by circulating the air through another filter, treating the air with a photocatalyst, removing humidity from the air, and/or the like.

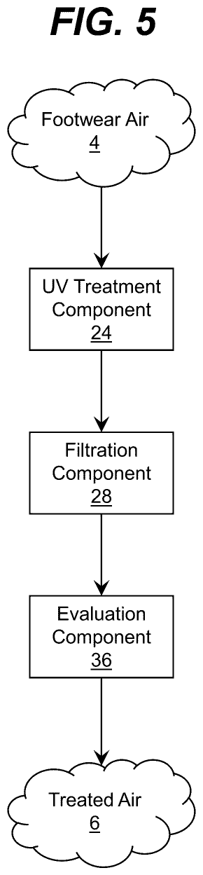

FIG. 5 shows an illustrative flow diagram according to embodiments. In general, during treatment, air 4 within the footwear is circulated so that it is exposed to ultraviolet light by an ultraviolet treatment component 24 and filtered by a filtration component 28. While the ultraviolet treatment is shown occurring prior to the filtering, it is understood that this is only illustrative, and the two can occur in any order, including concurrently. Furthermore, while only a single ultraviolet treatment and filtering are shown, it is understood that the air can undergo multiple ultraviolet treatments and/or filterings, each of which can be configured to target a different organism, molecule, and/or the like.

Embodiments can include an evaluation component 36, which can evaluate the treated air 6 for an effectiveness of the treatment. In embodiments, the treated air is recirculated throughout the footwear and/or a surrounding environment (e.g., within the treatment housing 40), and therefore is reevaluated as footwear air 4. The computer system 22 can perform the treatment for a predetermined amount of time. In embodiments the computer system 22 can stop a treatment when the treated air 6 and/or the footwear air 4 is indicated as having a sufficiently low level of the target organism(s) and/or compound(s).

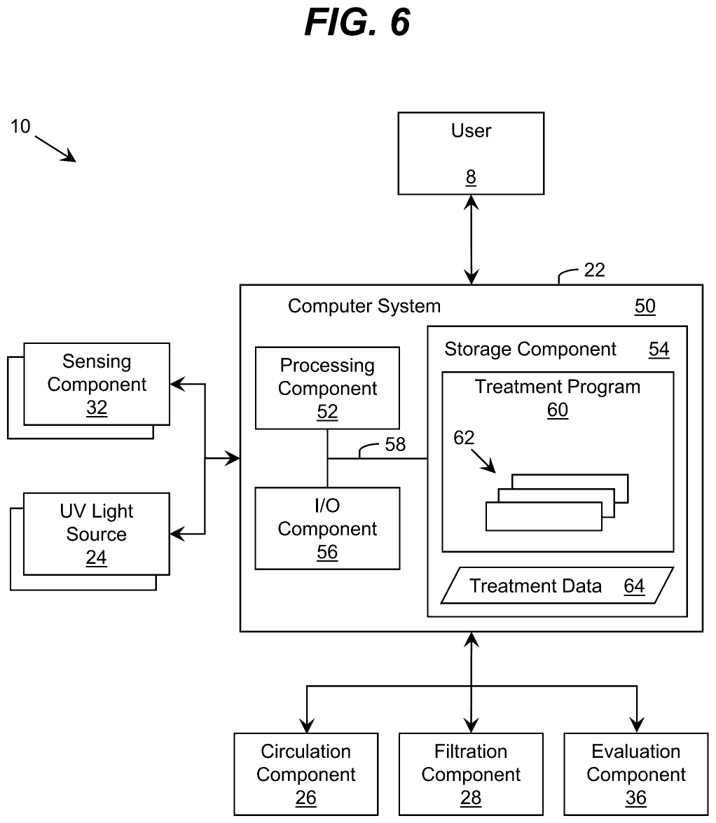

FIG. 6 shows still another illustrative footwear treatment system 10 according to an embodiment. To this extent, the footwear treatment system 10 includes a computing unit 22, which is implemented as a computer system 50 that can perform a process described herein in order to treat footwear. In particular, the computer system 50 is shown including a treatment program 60, which makes the computer system 50 operable to treat footwear by performing a process described herein.

The computer system 50 is schematically illustrated as including a processing component 52 (e.g., one or more processors), a storage component 54 (e.g., a storage hierarchy), an input/output (I/O) component 56 (e.g., one or more I/O interfaces and/or devices), and a communications pathway 58. In general, the processing component 52 executes program code, such as the treatment program 60, which is at least partially fixed in storage component 54. While executing program code, the processing component 52 can process data, which can result in reading and/or writing transformed data from/to the storage component 54 and/or the I/O component 56 for further processing. The pathway 58 provides a communications link between each of the components in the computer system 50. The I/O component 56 can comprise one or more human I/O devices, which enable a human user 8 to interact with the computer system 50 and/or one or more communications devices to enable a system user 8 (e.g., a portable computing device of a human user, such as a mobile phone executing an app) to communicate with the computer system 50 using any type of communications link. To this extent, the treatment program 60 can manage a set of interfaces (e.g., graphical user interface(s), application program interface, and/or the like) that enable human and/or system users 8 to interact with the treatment program 60 and the treatment data 64. Furthermore, the treatment program 60 can manage (e.g., store, retrieve, create, manipulate, organize, present, etc.) the treatment data 64 using any solution.

In any event, the computer system 50 can comprise one or more general purpose computing articles of manufacture (e.g., computing devices) capable of executing program code, such as the treatment program 60, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, the treatment program 60 can be embodied as any combination of system software and/or application software.

Furthermore, the treatment program 60 can be implemented using a set of modules 62. In this case, a module 62 can enable the computer system 50 to perform a set of tasks used by the treatment program 60, and can be separately developed and/or implemented apart from other portions of the treatment program 60. As used herein, the term “component” means any configuration of hardware, with or without software, which implements the functionality described in conjunction therewith using any solution, while the term “module” means program code that enables a computer system 50 to implement the actions described in conjunction therewith using any solution. Regardless, it is understood that two or more components, modules, and/or systems may share some/all of their respective hardware and/or software. Furthermore, it is understood that some of the functionality discussed herein may not be implemented or additional functionality may be included as part of the computer system 50.

When the computer system 50 comprises multiple computing devices, each computing device can have only a portion of the treatment program 60 fixed thereon (e.g., one or more modules 62). In embodiments, the computer system 50 can comprise a computing unit associated with the housing 14 and a portable computing unit, such as a mobile phone, which is executing an app installed thereon for enabling a user 8 to monitor, evaluate, manage, and/or the like, the footwear treatment system 10.

However, it is understood that the computer system 50 and the treatment program 60 are only representative of various possible equivalent computer systems that may perform a process described herein. To this extent, in other embodiments, the functionality provided by the computer system 50 and the treatment program 60 can be at least partially implemented by one or more computing devices that include any combination of general and/or specific purpose hardware with or without program code. In each embodiment, the hardware and program code, if included, can be created using standard engineering and programming techniques, respectively.

Regardless, when the computer system 50 includes multiple computing devices, the computing devices can communicate over any type of communications link. Furthermore, while performing a process described herein, the computer system 50 can communicate with one or more other computer systems using any type of communications link. In either case, the communications link can comprise any combination of various types of optical fiber, wired, and/or wireless links; comprise any combination of one or more types of networks; and/or utilize any combination of various types of transmission techniques and protocols.

As used herein, unless otherwise noted, the term “set” means one or more (i.e., at least one) and the phrase “any solution” means any now known or later developed solution. The singular forms “a,” “an,” and “the” include the plural forms as well, unless the context clearly indicates otherwise. Additionally, the terms “comprises,” “includes,” “has,” and related forms of each, when used in this specification, specify the presence of stated features, but do not preclude the presence or addition of one or more other features and/or groups thereof.

As also used herein, a layer is a transparent layer when the layer allows at least ten percent of radiation having a target wavelength, which is radiated at a normal incidence to an interface of the layer, to pass there through. Furthermore, as used herein, a layer is a reflective layer when the layer reflects at least ten percent of radiation having a target wavelength, which is radiated at a normal incidence to an interface of the layer. In an embodiment, the target wavelength of the radiation corresponds to a wavelength of radiation emitted or sensed (e.g., peak wavelength +/− five nanometers) by an active region of an optoelectronic device during operation of the device. For a given layer, the wavelength can be measured in a material of consideration and can depend on a refractive index of the material.

It is understood that, unless otherwise specified, each value is approximate and each range of values included herein is inclusive of the end values defining the range. Terms of degree such as “generally,” “substantially,” “about,” and “approximately” as used herein mean a reasonable amount of deviation of the modified term such that the end result is not significantly changed. For example, these terms can be construed as including a deviation of at least +/−0.5% of the modified term if this deviation would not negate the meaning of the word it modifies. In a more particular example, the term “approximately” is inclusive of values within +/− ten percent of the stated value, while the term “substantially” is inclusive of values within +/− five percent of the stated value when these deviations would not negate the meaning of the word each term modifies. Unless otherwise stated, two values are “similar” when the amount of deviation between the two values does not significantly change the result. In a more particular example, two values are similar when the smaller value is within +/− twenty-five percent of the larger value. A value, y, is on the order of a stated value, x, when the value y satisfies the formula 0.1x≤y≤10x.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A footwear treatment system comprising:
a pair of elongate supports, each elongate support including a cylindrical footwear extension region configured to be inserted into and hold a footwear item in an approximately vertical orientation;
at least one ultraviolet light source configured to emit ultraviolet light directed into an interior of the footwear item using the corresponding elongate support;
an air circulation component configured to circulate air within the interior of the footwear item using the corresponding elongate support; and
at least one filtration component configured to filter the circulating air.

2. The system of claim 1; wherein the cylindrical footwear extension region of each elongate support includes a hollow interior, and wherein at least a portion of a surface defining the hollow interior reflects the ultraviolet light.

3. The system of claim 1, further comprising a computing unit for controlling operation of the at least one light source and the at least one air circulation component.

4. The system of claim 1, further comprising at least one footwear sensor for detecting a presence of the footwear item on each of the pair of elongate supports.

5. The system of claim 1, further comprising a treatment housing, wherein the pair of elongate supports is located within an interior of the treatment housing.

6. The system of claim 5, further comprising:
a door configured to enable selective access to and enclosure of the interior of the treatment housing; and
at least one door sensor for determining whether a door for the treatment housing is open or closed.

7. The system of claim 1, further comprising a photocatalyst coating at least one surface of the footwear support structure.

8. The system of claim 1, further comprising a structure on which each elongate support is removably mounted, wherein a mounting location on the structure for each elongate support includes a communications and/or control interface.

9. The system of claim 1, further comprising at least one humidity sensor for detecting a humidity level in the footwear item.

10. The system of claim 1, further comprising at least one output device for communicating at least one of: a treatment status, a warning, or an error condition, for use by a user.

11. A footwear treatment system comprising:
an elongate support structure including a plurality of elongate supports, wherein each elongate support is configured to support one of a plurality of footwear items;
at least one ultraviolet light source configured to emit ultraviolet light directed into an interior of each of the plurality of footwear items using the elongate support structure;
an air circulation component for circulating air within the interior of each of the plurality of footwear items using the elongate support structure; and
a computing unit configured to control operation of the at least one light source and the air circulation component.

12. The system of claim 11, further comprising a filtration component for filtering the circulating air.

13. The system of claim 11, further comprising at least one footwear sensor for detecting a presence of a footwear item on each of the plurality of elongate supports, wherein the computing unit is configured to adjust operation of the at least one light source or the air circulation component based on data acquired by the at least one footwear sensor.

14. The system of claim 11, further comprising a treatment housing, wherein the elongate support structure is located within an interior of the treatment housing.

15. The system of claim 14, wherein the treatment housing includes:
a door configured to enable selective access to the interior of the treatment housing and enclosure of the interior of the treatment housing; and
a door sensor configured to detect whether the door is open or closed, wherein the computing unit is further configured to adjust operation of the at least one light source or the air circulation component based on data acquired by the door sensor.

16. The system of claim 11, further comprising an evaluation component configured to evaluate the circulating air for a presence of at least one compound associated with odor, wherein the computing unit is further configured to adjust operation of the at least one ultraviolet light source based on data acquired by the evaluation component.

17. The system of claim 11, further comprising at least one odor sensor configured to detect a presence of at least one of: a microorganism or a compound associated with odor, wherein the computing unit is further configured to adjust operation of the at least one ultraviolet light source based on data acquired by the at least one odor sensor.

18. The system of claim 11, further comprising at least one humidity sensor configured to detect a humidity level in the footwear item, wherein the computing unit is further configured to adjust operation of the at least one ultraviolet light source and the air circulation component based on data acquired by the at least one humidity sensor.

19. The system of claim 11, further comprising at least one output device operated by the control unit, wherein the control unit operates the at least one output device to communicate at least one of: a treatment status, a warning, or an error condition, for use by a user.

20. A method of treating footwear, the method comprising:
circulating with a fan air through a pair of hollow cylindrical elongate support, each of which is supporting a footwear item in an approximately vertical orientation;
emitting ultraviolet light from at least one ultraviolet light source into an interior of each footwear item using the corresponding elongate support; and
controlling the circulating and emitting based on at least one of: an amount of time, a treatment dose, a detected presence of a microorganism or a compound associated with odor, a detected humidity level, or a detected status of a door to a treatment housing in which the elongate support is located.
